(19) United States
(12) Patent Application Publication
(10) Pub. No.: US 2022/0160893 A1
(43) Pub. Date: May 26, 2022

(54) Gene Therapies, Systems, and Methods for Monitoring

(71) Applicant: Complete Medical Solutions, LLC, Ponte Vedra Beach, FL (US)

(72) Inventor: Charles Richardson, Ponte Vedra Beach, FL (US)

(73) Assignee: Complete Medical Solutions, LLC, Ponte Vedra Beach, FL (US)

(21) Appl. No.: 17/104,231

(22) Filed: Nov. 25, 2020

(51) Int. Cl. A61K 48/00; C12N 15/86

(52) U.S. Cl. CPC A61K 48/005; C12N 2750/14143; A61K 48/0075; C12N 15/86

(57) ABSTRACT

The disclosure relates to a modified adeno-associated virus (AAV) vector for treating various pathologies. The modified AAV vector may include transfected Claudin genes for use in treating those pathologies via the disclosed gene therapy. The disclosure also relates to the methods of preparing, administering, and testing the disclosed genetic therapies. Furthermore, the disclosure relates to systems and methods for monitoring, locally or remotely, a patient's medical condition and the efficacy of an administered genetic therapy. The system and methods also disclose adjusting the genetic therapies based on established operating parameters.

### FIELD OF INVENTION

The present invention relates generally to the use of gene therapy to treat disease. More particularly, the present disclosure relates to the products and processes implemented to provide treatments for targeted diseases.

### BACKGROUND OF THE INVENTION

One such disease that may be treated with gene therapy may include diabetic retinopathy, which causes high levels of glucose in the retina. Through a variety of mechanisms, increased glucose levels in the retina causes gradual damage to the retinal vasculature and eventually compromises retinal perfusion. This results in upregulation of vascular endothelial growth factor (“VEGF”) and other hypoxia-regulated genes. The increased levels of VEGF cause leukostasis, which worsens perfusion of the retina by occluding some retinal vessels. As diabetic retinopathy worsens, VEGF becomes the driver of the disease and results in the acceleration of the disease's progression.

Because the early stages of the disease progress so slowly, there are no good models of that part of the disease, but there are models of its later stages. The major problem in late-stage disease is that, as the VEGF levels continue to rise, they cause retinal vascular leakage and diabetic macular edema (“DME”), which result in the reduction of vision.

While intraocular injections of anti-VEGF antibodies have provided benefit in DME, they must be administered repeatedly and are not effective in many patients. Repeated administrations of small-molecule drugs can be costly, dangerous, and ineffective. Furthermore, the administration of such small-molecule drugs may only target disease symptoms and result in further complications. Although diabetic retinopathy is discussed in detail, any number of diseases could be substituted in this discussion.

What is needed then are improved methods for treating diseases and administering genetherapies.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one embodiment of the present invention, an adeno-associated virus (AAV) genome is disclosed that includes at least one inverted terminal repeat, a promotor, and either a Claudin-5 gene or a Claudin-3 gene. Another embodiment of the present invention includes a method of administering a gene therapy for treating diabetic macular edema, wherein an adeno-associated virus (AAV) is obtained and transfected with a Claudin-5 gene and then administered subretinally. The AAV is an attachment (does not necessarily have to be attached, i.e., it can be loosely connected) and administered through an appendage, usually through a needle. Therefore, any method may be utilized to get the AAV to attach, e.g., drop, needle, any access modality.

One aspect of the disclosure is a method of preparing an adeno- associated virus vector for treating a patient. A Claudin gene is inserted into the genome of the adeno-associated virus.

In another aspect of the disclosure, various Claudin and adeno-associated viruses are disclosed.

Another aspect of the disclosure is the formulations and compositions used for gene therapies.

In another aspect of the disclosure, a method of administering a modified adeno-associated virus vector to a patient is included. The method includes preparing the modified adeno-associated virus vector and administering the adeno-associated virus vector to a patient orally, topically, or via injection.

Another aspect of the disclosure is the testing of novel modified adeno- associated virus vectors for clinical efficacy using transgenic mice expressing symptoms similar to those of diabetic retinopathy.

Aspects of the disclosure also contain a discussion of the treatment of retinal diseases using the modified adeno-associated viruses discussed herein.

In another aspect of the disclosure, examples of treatments using the modified adeno-associated virus are disclosed.

Another aspect of the disclosure is the use of various modified adeno- associated virus vectors for treating vascular, skin, and inflammatory diseases as well as cancer.

Another aspect of the disclosure is systems and methods for analyzing the signals or other feedback generated by biological tissues for determining the appropriate timing, dosage, methods, and compositions when treating with gene therapies.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1a provides an exemplary embodiment of a modified adeno-associated virus (“AAV”) genome with an inserted transgene.

FIGS. 1b-1d provide exemplary embodiments of various modified AAV genomes.

FIG. 2 is an exemplary embodiment of a system for monitoring patients and gene therapies.

FIG. 3 is an alternative embodiment of a system for remotely monitoring patients and genetherapies.

### DETAILED DESCRIPTION

While the making and using of various embodiments of the present invention are discussed in detail below, it should be appreciated that the present invention provides many applicable inventive concepts that are embodied in a wide variety of specific contexts.

The specific embodiments discussed herein are merely illustrative of specific ways to make and use the invention and do not delimit the scope of the invention. Those of ordinary skill in the art will recognize numerous equivalents to the specific compositions and methods described herein. Such equivalents are considered to be within the scope of this invention and are covered by the claims.

The present invention is directed to systems and methods for preparing and administering gene therapies. In diabetic patients, hyperglycemia can result in a number of health complications both in the short term and over years. One serious result of sustained hyperglycemia can include damage to the patient's vasculature and, consequently, altered perfusion of the surrounding tissues. Decreased retinal perfusion as a result of damage to the retinal vasculature begins a cascade of regulation events that progresses diabetic retinopathy and may lead to blindness in the patient.

The present disclosure relates, in part, to the adverse secondary effects of the challenges in regulating blood glucose levels. This is especially true for patients who have developed insulin resistance or intolerance as increasing doses of insulin to the patient is not effective or practical. Although the control of glucose is often the primary target of diabetes management and corresponding therapies, the present disclosure, in part, is directed to the management of the secondary pathologies resulting from the sustained high levels of blood glucose.

These secondary pathologies are not limited to diabetic patients. Accordingly, this disclosure is not limited to the preparation and use of gene therapies for diabetic patients. A second non-limiting example includes macular degeneration, which can be a result of a similar vascular pathology with regards to diabetic retinopathy. Although, these examples will be discussed in more detail, the following disclosure is not to be construed to be only relevant to these two diseases, but may be used to treat a variety of diseases resulting from vascular pathologies that lead to various negative physiological and health outcomes, as well as non-vascular diseases.

The present disclosure addresses an unmet clinical need for effective, safe, and minimally invasive therapy in treating vascular pathologies. Various terms and methods will be described herein and will retain their common meaning as known by one of skill in the art unless otherwise indicated. Conventional techniques and methods in molecular biology are described herein and are provided in specific examples. However, one of skill in the art will recognize that in some instances, various other techniques, formulations, assays, and other components may be substituted to achieve similar results.

Some of the terms discussed herein will be listed below. As used herein, “VEGF” refers to vascular endothelial growth factor. VEGF is also known as vascular permeability factor and is a signal protein produced to stimulate the formation of blood vessels.

As used herein, “gene therapy” refers to a treatment that introduces target genes into a cell having defective, downregulated, missing, or non-existing genes, and as understood by one of skill in the art.

As used herein, “upregulation” refers to the process of increasing the production of or response to certain molecular stimuli, and as understood by one of skill in the art.

As used herein, “transgenic” refers to an organism containing genetic material that was artificially received or introduced into the organism, and as understood by one of skill in the art.

As used herein, “rho mice” refers to rhodopsin knockout mice that are used to model the function of the retina, and as understood by one of skill in the art.

As used herein, “AAV vector” refers to the adeno-associated virus used for transferring genetic material to a target organism, tissue, or cell, and as understood by one of skill in the art.

As used herein, “Claudin” refers to proteins that are a component in tight junctions establishing paracellular barriers in the intercellular space, and as understood by one of skill in the art.

As will be described herein, a composition, a method of producing the composition, and a method of administering the composition are disclosed.

Gene Therapy Vectors:

Gene therapy to correct defective genes requires efficient gene delivery and long-term gene expression. Realization of both goals with available vector systems has so far not been achieved. As a novel approach to solve this problem, a chimeric viral vector system has been developed that exploits favorable aspects of both adenoviral and retroviral vectors. In this schema, adenoviral vectors induce target cells to function as transient retroviral producer cells in vivo. The progeny retroviral vector particles can then effectively achieve stable transduction of neighboring cells. In this system, the nonintegrative adenoviral vector is rendered functionally integrative via the intermediate generation of an induced retroviral producer cell. Such chimeric vectors may now allow realization of the requisite goals for specific gene therapy applications.

In some embodiments, the gene therapy disclosed herein comprises an AAV vector modified to include a Claudin gene. AAV vectors are effective because of their ability to infect both dividing and non-dividing cells and because of demonstrated persistent expression inside of a cell after infection. Likewise, AAV vectors have been found to be non-pathogenic and, therefore, are effective without causing harm to the target cell or activating a significant immune response to infection by the AAV vector.

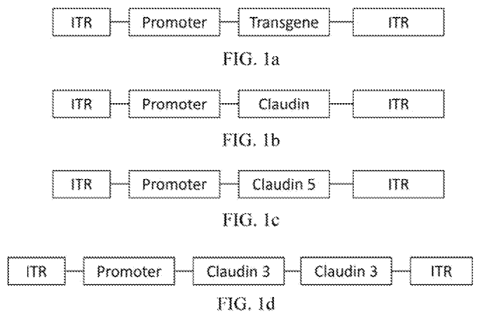

AAV vectors generally include single-stranded DNA, wherein the genome includes inverted terminal repeats surrounding two open reading frames. The inverted terminal repeats include base pair sequences forming a palindrome that facilitate pairing of the base pairs to form T-shaped hairpin structures in the DNA. The use a terminal repeat may occur all the way through (a 3, 5, or combination of both). In some embodiments, the Claudin gene may be inserted into the AAV genome via methods, which will be discussed later in further detail. FIGS. 1a-1d demonstrate exemplary embodiments of a modified AAV vector's genome including inserted transgenes.

AAV vectors also include a capsid, or a protein shell, which encloses the genome of the vector, including the inverted terminal repeats, the reading frames, and the native or inserted genes. The capsid also may act to interact with and deliver the genome to the target cell or tissues. The capsid may be modified to target specific cells or tissues and will be discussed later in further detail.

Various AAV vectors may be utilized in preparing an effective gene therapy compound. In some embodiments, a recombinant adeno-associated virus (“rAAV”) vector may be transfected with a Claudin gene. rAAV vectors have been shown to effectively transduce muscle, liver, brain, retina, and lung tissue. In other embodiments, a trans-splicing adeno-associated virus (“tsAAV”) may be transfected with a Claudin gene. tsAAV vectors have been shown to effectively package and carry increased genetic material and therefore has greater capacity. In other embodiments, a self-complimentary adeno-associated virus (“scAAV”) may be transfected with a Claudin gene. scAAV vector may be useful in increasing the rate of transduction of target cells and tissues.

Furthermore, AAV vectors may include at least 12 human serotypes that may be useful in various settings for use of the disclosed gene therapy. For example, specific existing AAV serotypes may be better suited for targeting specific cells or tissues. AAV1 has been shown to effectively transduce into the central nervous system, cardio, retinal, and skeletal muscle cells and tissue. AAV2 has been shown to effectively transduce into the central nervous system, renal, photoreceptor, and retinal cells and tissue. AAV4 has been shown to effectively transduce into the central nervous system, lung, and retinal cells and tissue. AAV5 has been shown to effectively transduce into the central nervous system, lung, photoreceptor, and retinal cells and tissue. AAV6 has been shown to effectively transduce into lung and skeletal muscle cells and tissue. AAV7 has been shown to effectively transduce into liver and skeletal muscle cells and tissue. AAV8 has been shown to effectively transduce into the central nervous system, cardio, liver, pancreatic, photoreceptor, retinal, and skeletal muscle cells and tissue. AAV9 has been shown to effectively transduce into the central nervous system, cardio, liver, lung, and skeletal muscle cells and tissue.

In some embodiments, the preparation of a gene therapy using a Claudin gene may include removing a portion of the existing genetic information from an AAV vector. Removal of certain portions of existing viral genetic information may prevent certain unintended replications or other side effects from occurring when human cells are transfected with the AAV vector, either in vivo or in vitro. The AAV vector, unaltered, contains genetic information encoding capsid proteins and Rep proteins. However, in some embodiments, it may be advantageous to retain the genetic information encoding the capsid and Rep proteins.

Isolation and Characterization of Claudin Genes:

Disruption of the blood-retina barrier (“BRB”) is an early phenomenon in preclinical diabetic retinopathy (“PCDR”). Two vascular permeability pathways may be affected, the paracellular pathway involving endothelial cell tight junctions, and the endothelial transcellular pathway mediated by endocytotic vesicles (caveolae). The relative contribution of both pathways to vascular permeability in PCDR is unknown.

Transcription levels were compared in entire rat retina of genes related to these pathways between control conditions and after 6 and 12 weeks of streptozotocin- induced diabetes, as well as in bovine retinal endothelial cells (“BREC”) exposed to VEGF and bovine retinal pericytes (“BRPC”), using quantitative RT-PCR. To confirm endothelial-specificity, immunohistochemical staining was performed in rat retina, and mRNA transcript levels were compared between BRECs and BRPCs. mRNA and protein of most paracellular transport-related genes were specifically expressed by retinal endothelial cells, whereas vesicle transport-related mRNA and proteins were present in various retinal cell types, including endothelial cells. Expression of selected endothelial cell tight junction genes and particularly that of occludin and Claudin-5 was reduced in the diabetic retina and in BRECs after exposure to VEGF. Expression of 6 out of 11 vesicular transport-related genes was upregulated after induction of diabetes. Of these, only plasmalemma vesicle-associated protein (PV-1) was exclusively expressed in BRECs and not in BRPCs. PV-1 transcription was markedly induced in diabetic retina and by VEGF in BRECs.

The endothelial tight junction genes occludin and Claudin-5 showed a transient downregulation, and long-term upregulation was observed in diabetic retina and VEGF-induced expression in BRECs of the vesicular transport-related genes caveolin-1 and PV-1. The altered gene expression profiles observed in the study suggest a transient induction of the paracellular pathway and prolonged involvement of transcellular endothelial transport mechanisms in the increased permeability of retinal capillaries in PCDR.

Claudins are integral 4-pass transmembrane proteins exclusively located at tight junctions and, in contrast to both junctional adhesion molecules (“JAMs”) and occludin, are sufficient for tight junctions' induction. In mammals, the Claudin family is composed of 27 known members that display tissue specific expression patterns and different functions. While some Claudins, e.g. Claudin-1 and Claudin-3 form paracellular barriers, other Claudins, e.g. Claudin-2 or Claudin-16, form paracellular pores allowing for controlled diffusion of ions and water via the tight junctions. Each tight junction is established by a combination of different Claudins and therefore the tightness of individual strands of the tight junctions is determined by the combination and mixing ratio of Claudins.

More specifically, Claudin-5 is an endothelial cell-specific component of tight junction strands and is highly expressed in blood brain barrier tight junctions of rodents, zebrafish, nonhuman primates, and humans. To clarify expression and function of Claudin-3 in the tight junctions of the brain barriers, Claudin-_,_mice were backcrossed to C57BL/6 background mice allowing the study of brain barrier function in health and neuroinflammation in experimental autoimmune encephalomyelitis (“EAE”). Unexpectedly, Claudin-3 is not expressed in tight junctions of mouse blood brain barrier endothelial cells, in vitro and in vivo. Prior evidence supporting Claudin-3 expression at the blood brain barrier is due to cross-reactivity of anti-Claudin-3 antibodies with an unknown endothelial junctional antigen detectable in blood brain barrier tight junctions of Claudin-_,_mice. The presence of Claudin-3 in the epithelial tight junctions of the blood cerebrospinal fluid barrier was confirmed. Notably, in C57BL/6 mice lacking Claudin-3, no impairment of the brain barrier properties in vivo was observed.

The insertion of a Claudin gene may occur either directly into an AAV vector's genome with the AAV vector's existing genetic information, or the Claudin gene may replace the genetic information that was previously discussed. Various Claudin genes may be inserted into the AAV vector for use in gene therapy. For example, Claudin-3 and Claudin-5 may be used specifically in treating diabetic retinopathy and macular degeneration. In some embodiments, both the Claudin-3 gene and the Claudin- 5 gene may be inserted into a single AAV vector. In other embodiments, any one of the Claudin-1 gene, Claudin-2 gene, Claudin-3 gene, Claudin-4 gene, Claudin-5 gene, Claudin-6 gene, Claudin-7 gene, Claudin-8 gene, Claudin-9 gene, Claudin-10 gene, Claudin-11 gene, Claudin-12 gene, Claudin-13 gene, Claudin-14 gene, Claudin-15 gene, Claudin-16 gene, Claudin-17 gene, Claudin-18 gene, Claudin-19 gene, Claudin-20 gene, Claudin-21 gene, Claudin-22 gene, Claudin-23 gene, Claudin-24 gene, Claudin-25 gene, Claudin-26 gene, or Claudin-27 gene may be inserted into a single AAV vector.

In other embodiments, the Claudin-3 gene may be inserted into an AAV vector in combination the Claudin-1 gene, Claudin-2 gene, Claudin-4 gene, Claudin-5 gene, Claudin-6 gene, Claudin-7 gene, Claudin-8 gene, Claudin-9 gene, Claudin-10 gene, Claudin-11 gene, Claudin-12 gene, Claudin-13 gene, Claudin-14 gene, Claudin-15 gene, Claudin-16 gene, Claudin-17 gene, Claudin-18 gene, Claudin-19 gene, Claudin-20 gene, Claudin-21 gene, Claudin-22 gene, Claudin-23 gene, Claudin-24 gene, Claudin-25 gene, Claudin-26 gene, or Claudin-27 gene. In other embodiments, the Claudin-5 gene may be inserted into an AAV vector in combination the Claudin-1 gene, Claudin-2 gene, Claudin-3 gene, Claudin-4 gene, Claudin-6 gene, Claudin-7 gene, Claudin-8 gene, Claudin-9 gene, Claudin-10 gene, Claudin-11 gene, Claudin-12 gene, Claudin-13 gene, Claudin-14 gene, Claudin-15 gene, Claudin-16 gene, Claudin-17 gene, Claudin-18 gene, Claudin-19 gene, Claudin-20 gene, Claudin-21 gene, Claudin-22 gene, Claudin-23 gene, Claudin-24 gene, Claudin-25 gene, Claudin-26 gene, or Claudin-27 gene. In alternative embodiments, any one of Claudin-1 through Claudin-27 genes may be inserted in combination with any other of the Claudin-1 through Claudin-27 genes into an AAV vector.

The Claudin-5 gene in an AAV vector is especially suited for treatment of diabetic retinopathy as the Claudin-5 protein is a major component of tight junctions between vascular endothelial cells.

Methods for Preparing a Gene Therapy:

Various methods for preparing the effective gene therapy will be described herein. One method includes preparing the rAAV DNA to include the transgene. In one embodiment, the transgene to be inserted into the rAAV DNA includes any one of the Claudin genes. More specifically, the Claudin-3 or Claudin-5 gene may be inserted into the rAAV DNA, where the Claudin-3 or Claudin-5 gene are flanked by the inverted terminal repeats and open reading frames of the rAAV DNA and includes promoters for gene replication and transcription. If there is a gene and something is put with it to cause a reaction, it can promote a quicker result.

Likewise, a plasmid is either prepared or obtained, which contains the Rep and capsid genes of the AAV. The Rep and capsid genes contained on the plasmid must be flanked by the inverted terminal repeats in order for the Rep and capsid proteins to be packaged in the final rAAV vector. In some embodiments, the preparation may also include an Adenovirus helper virus, which includes components necessary for proper replication and assembly of the modified rAAV vector containing the Claudin-3 or Claudin-5 gene. In other embodiments, the preparation may include only the Adenovirus helper genes in plasmid format rather than the full Adenovirus helper virus.

Once the preparation is ready, these elements are introduced into a packaging cell line where the modified rAAV vector is produced and assembled. In some embodiments, the HEK293 cell line is used to produce the modified rAAV vector. The modified rAAV vector is then isolated from the packaging cell line.

Once the rAAV vector has been isolated, the rAAV vector may be stored in a formulation or may be prepared for administration. Some of these formulations may include aqueous solutions, buffers, solvents, or any other compound known by one of skill in the art for storing or administering viral vectors.

Compositions and Formulations:

The present disclosure includes the various compositions making up the gene therapy. Such compositions may include vectors, proteins, nucleic acids, carriers such as solvents, solutions, coatings, buffers, and any other component known by one of skill in the art for storing and administering pharmaceutical compositions. In some embodiments, the effective treatment may be administered to a patient in conjunction with other solutions and compositions even if the gene therapy and the other solution and compositions are never combined ex vivo.

The compositions and formulations may vary depending on the method of administering the gene therapy. For example, the formulation of the gene therapy will vary when administered via injections directly to the site versus topical administration, oral, etc.

Glucocorticoids may be used in conjunction with the disclosed gene therapies. Glucocorticoids may be used to restore vascular barrier properties in the retina. Glucocorticoids are useful in preserving the integrity of the blood-brain barrier in the treatment of brain tumors, and these steroids show similar effects on the retinal vasculature suggesting their potential usefulness in treating diabetic retinopathy. Glucocorticoids may act by both suppressing inflammation and by directly affecting the endothelial cells by regulating phosphorylation, organization, and content of tight junction proteins.

By way of example, tight junctions between vascular endothelial cells help to create the blood-brain and blood-retinal barriers. Breakdown of the retinal tight junction complex is problematic in several disease states including diabetic retinopathy. Glucocorticoids may restore and/or preserve the endothelial barrier to paracellular permeability. Glucocorticoid treatment of primary retinal endothelial cells increases content of the tight junction proteins occludin and Claudin-5, co-incident with an increase in barrier properties of endothelial monolayers. The glucocorticoid receptor antagonist RU486 reverses diabetes type I or type II with other chronic disease in both the glucocorticoid-stimulated increase in occludin content and the increase in barrier properties. Transcriptional activity from the human occludin and Claudin-5 promoters increases in retinal endothelial cells upon glucocorticoid treatment, and is dependent on the glucocorticoid receptor (“GR”) as demonstrated by siRNA. Deletion analysis of the occludin promoter reveals a 205 bp sequence responsible for the glucocorticoid response. However, this region does not possess a canonical glucocorticoid response element and does not bind to the GR in a chromatinimmunoprecipitation(“ChlP”) assay.

Mutational analysis of this region revealed a novel 40 bp occludin enhancer element (OEE), containing two highly conserved regions of 10 and 13 base pairs, which is both necessary and sufficient for glucocorticoid-induced gene expression in retinal endothelial cells. These data suggest a novel mechanism for glucocorticoid induction of vascular endothelial barrier properties through increased occludin and Claudin-5 gene expression. Therefore, in some embodiments, glucocorticoids may be used in combination with the disclosed gene therapies.

Methods for Administering GeneTherapy:

Using the above compositions and formulations, the modified AAV vectors may be administered to a patient. Some of these methods include administration by injection. When administering via injection, the modified AAV vector and its carrying compounds may be injected directly into the tissue to be treated. This may include injections into the specific vasculature that is diseased, when the vessels are the target tissue. For example, subretinal injections may be employed for treating the vasculature for the retina. Likewise, the injection may be administered proximate the target tissue. An example of this may include intravitreal injections for treating the vasculature for the retina.

Other methods of administering the modified AAV vector may include oral administration. In some embodiments, the modified AAV vector may be prepared in an oral dose to be administered to the patient. This method of administration has been found to increase compliance and disseminate widely through the body. However, some disadvantages might exist, for example, in treatments where a specific tissue is targeted, the oral administration may not provide the appropriate concentration of the therapy to the target tissue, or may require repeated administration or high doses to achieve the desired pharmacological effectiveness.

An alternative method of administering the modified AAV vector may include topical administration. In some embodiments, the modified AAV vector may be prepared in a topical treatment to be administered to the patient.

Methods of Testing Gene Therapy:

The present disclosure furthermore discusses methods of testing gene therapy using Claudin AAV vectors in treating vascular diseases. For example, in testing the effectiveness of a gene therapy for diabetic retinopathy, transgenic mice may be utilized to determine the effectiveness of any of the disclosed gene therapy compounds. Certain transgenic mice may provide analogous pathophysiological conditions to those experienced during diabetic retinopathy. Transgenic mice that express VEGF in the retina have excessive vascular leakage in the retina analogous to patients with diabetic macular edema (“DME”). Mice in which the rhodopsin promoter drives expression of VEGF in the retina (e.g., rhoNEGF mice) model moderately severe disease and therefore provide a model for moderate DME. Double transgenic mice with doxycycline-inducible expression produce very high levels of VEGF in the retina and model severe DME. Transgenic mice receive clinically effective amounts of the Claudin AAV vectors in order to treat diabetic macular edema.

As will be discussed hereafter, a system and method will be disclosed for monitoring the performance of administered gene therapies.

Retinal Gene Therapy:

The vertebrate neural retina is composed of several layers and distinct cell types. A number if these cell types are implicated in retinal diseases, including retinal ganglion cells, which degenerate in glaucoma, the rod and cone photoreceptors, which are responsive to light and degenerate in retinitis pigmentosa, macular degeneration, and other retinal diseases and the retinal pigment epithelium, which supports the photoreceptors and is also implicated in retinitis pigmentosa and macular degeneration. In retinal gene therapy, AAV vectors are capable of transducing these various cell types by entering the cells and expressing the therapeutic DNA sequences. Since the cells of the retina are non-dividing, the AAV vector continues to persist and provide expression of the therapeutic DNA sequence over an extended period of time that can last several years.

AAV vectors are capable of transducing multiple cell types within the retina. AAV serotype 2 can be administered either intravitreal or subretinal. Using the intravitreal administration, the AAV vector is injected in the vitreous humor of the eye. Using the subretinal route, the AAV is injected underneath the retina, taking advantage of the potential space between the photoreceptors and the retinal pigment epithelium layer in a short surgical procedure. Although subretinal administration is more invasive than the intravitreal administration, administered fluid is absorbed by the retinal pigment epithelium and the retina flattens in less than 14 hours without complications. Intravitreal AAV vectors target retinal ganglion cells and some Muller glial cells. Subretinal AAV vectors efficiently target photoreceptors and retinal pigment epithelium cells.

The various routes of administration lead to different cell types being transfected (e.g., different tropism) because the inner limiting membrane and the various retinal layers act as physical barriers for the delivery of drugs and vectors to the deeper retinal layers.

Thus, in some embodiments, the subretinal AAV vector administration is 5 to 10 times more efficient than delivery using the intravitreal route. [0076]An important factor in gene delivery is developing altered cell tropisms to narrow or broaden rAAV-mediated gene delivery and to increase its efficiency in tissues, including ocular tissue. Specific properties like capsid conformation, cell- targeting strategies can determine which cell types are affected and the efficiency of the gene transfer process. Different kinds of modifications may be undertaken. For example, modification by chemical, immunological, or genetic changes may enable to AAV2 capsid to interact with specific cell surface molecules.

Several natural-occurring serotypes of AAV have been isolated and have been found to successfully transduce retinal cells. Following intravitreal injection, only AAV serotypes 2 and 8 were capable of transducing retinal ganglion cells. Occasional Muller cells were transduced by AAV serotypes 2, 8, and 9. Following subretinal injections, serotypes 2, 5, 7, and 8 efficiently transduced photoreceptors, and serotypes 1, 2, 5, 7, 8, and 9 efficiently transduced retinal pigment epithelium cells.

An example of an engineered variant has been demonstrated to efficiently transduce Muller glia following intravitreal injection and has been used to rescue an animal model of aggressive, autosomal-dominant retinitis pigmentosa.

AAV vectors are also effective on the retina because the retina is immune- privileged. Thus, the retina does not experience a significant inflammation or immune response when AAV vectors are injected. Immune response to gene therapy vectors is what, in some cases, has caused attempts at gene therapy to fail. Likewise, re- administration of AAV vectors in large animals has been successful, indicating that no long-lasting immune response is mounted. In some cases, the subretinal route may be subject to a greater degree of immune privilege compared to the intravitreal route.

### EXAMPLE

A first example of a targeted genetic therapy is the treatment of inherited blindness as a result of inherited retinal degenerations. A modified AAV vector may be used to treat the subject eye. Treatment of the eye with genetic therapies is advantageous as the eye is a small organ requiring relatively small amounts of pharmaceutical compounds, is easily accessible for administration of the therapy, is immune-privileged as the blood-retinal barrier keeps the retinal environment isolated and reduces AAV dissemination throughout the patient's body, and is easily assessed for functionality using a range of clinically-based electrophysiological and psychophysiological tests.

Various AAV vectors may be employed in the treatment of inherited retinal degenerations. In one embodiment, an AAV vector may be transfected with the Claudin-5 gene to form a Claudin-5 AAV vector. The Claudin-5 AAV vector is administered to the degenerative retina via a single subretinal injection. The Claudin-5 AAV vector targets the retinal cells containing the corresponding targets of the capsids of the Claudin-5 AAV vector. The Claudin-5 AAV vector transduces the targeted retinal cells, which then produce Claudin-5. Claudin-5 is a junctional protein that is a major component of tight junctions between vascular endothelial cells. The Claudin-5 proteins will ameliorate the symptoms of retinal degeneration in specific instances in which the inherited degeneration is a result of macular edema (e.g., retinoschisis or retinitis pigmentosa), by enhancing the damaged or ineffective tight junctions of the vascular endothelial cells near the retina.

In another embodiment, the AAV vector may be transfected with the Claudin-3 gene to form a Claudin-3 AAV vector. The Claudin-3 AAV vector is similarly administered to the degenerative retina via a single subretinal injection. Similar variations may each be similarly prepared and administered to defective the degenerative retina, including, but not limited to: Claudin 1 AAV vector, Claudin 2 AAV vector, Claudin-3 AAV vector, Claudin 4 AAV vector, Claudin-5 AAV vector, Claudin 6 AAV vector, Claudin 7 AAV vector, Claudin 8 AAV vector, Claudin 9 AAV vector, Claudin 10 AAV vector, Claudin 11 AAV vector, Claudin 12 AAV vector, Claudin 13 AAV vector, Claudin 14 AAV vector, Claudin 15 AAV vector, Claudin 16 AAV vector, Claudin 17 AAV vector, Claudin 18 AAV vector, Claudin 19 AAV vector, Claudin 20 AAV vector, Claudin 21 AAV vector, Claudin 22 AAV vector, and Claudin 23 AAV vector.

Alternative Applications:

An alternative application of a targeted genetic therapy in the treatment of inherited blindness as a result of inherited retinal degenerations using a modified Claudin AAV vector. The modified Claudin AAV vector may be transfected and administered similarly as discussed in Example 1.

A second alternative application of a targeted genetic therapy is the treatment of photoreceptor cells of a patient's eye suffering from age-related macular degeneration. The treatment includes administering a modified AAV vector to or near the retina. In order to achieve the best possible transduction efficiency of photoreceptor cells using an AAV vector, a subretinal injection may be the best approach as it places the AAV vector immediately adjacent to the target photoreceptor cells. Traditional age- related macular degeneration is treated using frequent injections of recombinant protein into the eye, the use of the disclosed modified Claudin AAV vectors result in long-term disease management following a single administration of the modified Claudin AAV.

A third alternative application of a targeted genetic therapy in the treatment of retinal degenerations using a modified AAV vector includes the administration of an intravitreal injection of the modified AAV vector.

Because a subretinal injection creates a temporary retinal detachment and does carry certain risk for being relatively invasive, it may be advantageous to administer the modified AAV vector via an intravitreal injection. While in most cases the benefit to the patient from the subsequent gene supplementation by the AAV vector would surpass any effect the temporary retinal detachment might have, a less invasive administration via an AAV vector administered intravitreally may be preferable in some embodiments. The difficulty with an injection of the AAV vector to the vitreous of the eye is that the AAV vector must cross many layers of supporting neurons in order to reach the deeper retinal layers where the photoreceptors lie. In some embodiments, because of the small size of the AAV vector, the intravitreal injection of the modified AAV is still capable of reaching the targeted cells.

A fourth alternative application includes the treatment of metastatic brain tumors. Metastatic brain tumors are frequently observed in patients with lung, breast and malignant melanoma and are a severe complication of metastatic cancers. With improved primary cancer treatments, including surgery, radiation therapy and chemotherapy, patients are now living longer following initial treatment, compared with previous treatments. However, brain metastasis (“BM”) remains a significant clinical issue. Since BM represents a major therapeutic challenge, it is vital that the mechanisms of interaction between tumor cells and the blood-brain barrier (“BBB”), as well as the method by which tumor cells establish metastatic tumors in the brain, are understood. A key step in BM is the interaction and penetration of the BBB by cancer cells. The BBB consists of endothelial cells, pericytes, astrocytes and a number of molecular structures between these cells. The BBB relies on the tight junctions that are present between the endothelial cells of the brain capillaries to provide a closed environment for the brain. As previously discussed, tight junctions comprise a number of proteins, including occludin, Claudins and junctional adhesion molecules. Among them, Claudins are the key integral proteins that regulate BBB permeability. Claudin-5 not only regulates paracellular ionic selectivity, but also plays a role in the regulation of tumor cell motility. Thus, the disclosed gene therapies may likewise be used for treating or ameliorating BM. Effective treatment using Claudin-5 gene therapies may be achieved to help the control of BM.

In some alternative embodiments, the disclosed compounds and methods can be used to treat various other vascular diseases. Such diseases may include peripheral edema, pedal edema, lymphedema, pulmonary edema, and cerebral edema. The disclosed therapies may also be applied to a variety of other blood vessel pathologies and diseases.

Relatedly, the disclosed therapies may likewise be applied to various cardiac diseases and pathologies. For instance, the disclosed therapies may be administered to patients experiencing or at risk for congestive heart failure. Claudin-5 is transcriptionally downregulated resulting in dramatically reduced protein levels in human heart failure. Studies in mice have demonstrated that reduced Claudin-5 levels occur prior to cardiac damage and before reduced whole heart function. Therefore, Claudin-5 may be a useful early therapeutic target for human heart failure. Claudin-5 has been observed to localize to cardiomyocytes, endothelial cells, and a subset of fibroblasts in non-failing human heart sections. In isolated cardiomyocytes, the transmembrane Claudin-5 protein localizes in longitudinal striations in lateral membranes. In a failing heart, both cardiomyocyte and endothelial Claudin-5 localization is severely reduced, but Claudin-5 remains in fibroblasts. The absence of Claudin-5 is also correlated with the reduction of the endothelial cell marker CD31. Ephrin-81 localization, but not protein levels, is altered in failing hearts supporting that Claudin-5 is required for ephrin-81 localization. Thus, the administration of the disclosed therapies may likewise be used in treating and preventing congestive heart failure.

Relatedly, Claudin-5 also localizes to lateral membranes of murine cardiomyocytes at their junction with the extracellular matrix. Claudin-5 levels are specifically reduced in myocytes from a mouse model of muscular dystrophy with cardiomyopathy. Claudin-5 levels are reduced in at least 60% of patient samples compared with non-failing controls. Importantly, Claudin-5 reductions can be independent of connexin-43, which is a gap junction protein that is reduced in failing heart samples. Other cell junction proteins including alpha-catenin, beta-catenin, gamma-catenin, desmoplakin, and N-cadherin are reduced in only a small number of failing samples and only in combination with reduced Claudin-5 or connexin-43 levels. Reduced Claudin-5 levels can be present independently from dystrophin alterations, which are known to be capable of causing and resulting from cardiomyopathy. Thus, Claudin-5 may participate in the pathway to end-stage heart failure. Thus, the administration of the disclosed therapies may likewise be used in treating and preventing congestive heart failure. Furthermore, administration of the disclosed therapies may also be used in treating and preventing cardiomyopathy.

Other diseases may likewise be treated by the disclosed genetic therapies. For example, the disclosed therapies may be used to treat skin diseases such as ichthyosis, psoriasis, and bacterial infections.

In another embodiment, the disclosed genetic therapies may be used to treat various cancers such as invasive ductal cancer, prostatic adenocarcinoma, thyroid neoplasma, follicular adenoma, gastroesophageal reflux disease, and basaloid squamous carcinoma.

In other embodiments, the disclosed genetic therapies may be used to treat various inflammatory diseases such as morbus Crohn, collagenous colitis, multiple sclerosis, hereditary deafness, familial hypomagnesemia, cystic fibrosis, and clostridium perfingens enterotoxin.

In another alternative embodiment, the disclosed genetic therapies may be used to treat velocardiofacial syndrome.

In another alternative embodiment, the disclosed genetic therapies may be used to treat diseases or pathologies relating to the BBB. For example, the disclosed therapies may be used to treat concussions and Parkinson's disease.

Monitoring and Remote Monitoring of Gene Therapies:

One apparent difficulty in the use of gene therapies is the ability to determine when and how to administer the therapy. The present disclosure likewise discusses systems and methods that can be used to analyze the signals or other feedback generated by biological tissues for determining the appropriate timing, dosage, methods, and compositions when treating with gene therapies. The result of the analysis of the signals and feedback indicates the condition of the tissue, or a particular aspect thereof, and results obtained over time can alert a patient or health care professional to changes in that condition.

Generally, the methods are carried out by obtaining a recording of biological activity that includes multiple signaling events (e.g., periodic waveforms representative of the electrical activity), selecting one or more features of an event (e.g., its amplitude or frequency), and calculating an average value representing that event over a given number of selected multiples (e.g., 2-500 or 100-200). The calculation can be, for example, a calculation of an area under a curve or a defined portion of a curve, a frequency difference between first and second points, which may each be within consecutive events, an ascending slope from a nadir to an apex or a defined portion between the nadir and apex, a descending slope from an apex to a nadir or a defined portion between the apex and nadir, or a difference in amplitude between an apex and a nadir.

In one example, the recording of electrical activity obtained at a first point in time may be referred to as the “baseline recording.” Likewise, the average result of a first calculation performed on a multiple of events within the baseline recording may be referred to as the “first baseline template” (a second calculation giving rise to a second baseline template; a third calculation giving rise to a third baseline template; and so forth). The same process, or a substantially equivalent process, can be carried out on a recording of electrical activity obtained at a second and later point in time. The recording obtained at the second point in time may be referred to as the “captured recording,” and the average result of a first calculation performed on a multiple of events within the captured recording may be referred to as the “first captured template” (a second calculation giving rise to a second captured template; a third calculation giving rise to a third captured template; and so forth). Once a baseline and corresponding captured template have been obtained, the variance between them is calculated. Where more than one feature is analyzed, giving rise to a second set of templates, we may refer to a second variance (i.e., the variance between the second baseline template and the second captured template); a third variance; a fourth variance; and so forth.

As some features of the signaling events may be more indicative of the condition of the tissue than others, various embodiments (e.g., the systems, methods, and computer programs of the invention) can also include a step in which the variances are scaled or weighted. For example, where two features of a signaling event are analyzed, one can scale first and second variances by first and second weighting factors. The sum of the first and second scaled variances can then be calculated. The calculated sum represents a difference between the baseline and captured templates that indicates a change in the condition of the tissue over time within the same patient. The calculated result from the baseline template can be compared to the calculated result from the captured template, a difference indicating a change in the condition of the tissue.

[000102] Accordingly, in a specific embodiment, the disclosure relates a method for assessing a medical condition by: (1) obtaining a first baseline template corresponding to an average of results of a first calculation performed on a multiple of events within a first electrical signal acquired at a first time; (2) obtaining a first captured template corresponding to an average of results of the first calculation performed on a multiple of events within a second electrical signal acquired at a second time, which is later than the first time; (3) obtaining a second baseline template corresponding to an average of results of a second calculation performed on a multiple of events within the first electrical signal; (4) obtaining a second captured template corresponding to an average of results of the second calculation performed on a multiple of events within the second electrical signal; (5) calculating a first variance between the first baseline and captured templates; (6) calculating a second variance between the second baseline and captured templates; (7) optionally, scaling the first and second variances by first and second weighting factors, respectively, to produce first and second scaled variances; and (8) calculating a first sum of the first and second variances or, if scaled, of the scaled variances.

Where the first sum indicates that the variance between the baseline and captured templates is about 5-10%, the patient and/or a member of their health care team would be alerted that there is a change in the condition of the tissue that merits close observation and, if advisable, intervention. Intervention may be appropriate, for example, in view of the patient's overall medical condition and/or where other diagnostic tests or subjective input from the patient indicates as much. Accordingly, the methods, systems, and programs of the invention can include providing and assessing data obtained from the patient (e.g., by way of a questionnaire or interview) or other diagnostic tests. Where the first sum indicates that the variance between the baseline and captured templates is about 10-15%, the patient and/or a member of their health care team would be alerted that there is a substantial and probably seriously detrimental change in the condition of the tissue. For example, where the tissue being monitored is a transplanted heart, a variance of 10-15% (or more) indicates that the heart is in the process of rejection.

The teaching above assumes that the change in the electrical activity within the tissue is detrimental; that one or more of the features of the captured template are less desirable than those of the baseline template. The methods, systems, and programs of the invention may also reveal, however, an improvement or stabilization of a medical condition. For example, where one or more of the baseline templates are obtained from a healthy subject, a decrease in the variance between one or more of the baseline templates and one or more of the corresponding captured templates would indicate an improvement in the patient from whom the captured template was obtained. For example, a baseline template can be calculated based on a recording obtained from the patient being evaluated at an earlier time when the patient was in better health or from another patient or group of patients who are healthy or who have responded positively to a treatment or procedure.

Thus, whether the outcome indicates a detrimental, beneficial, or minimal change in a given patient's condition, one can generate or obtain threshold values that are indicative of a medical condition of interest. For example, one can obtain one or more baseline templates from a patient or group of patients having a particular medical condition. For example, the patient may be one who has received genetic therapies for treating diabetic retinopathy. In other instances, the patient may be one who has been diagnosed with a neurodegenerative disease (e.g., multiple sclerosis) that is in an early stage or who that has reached an advanced stage. In each instance, one can generate a baseline template from a patient of interest and compare this template to a captured template generated from the patient. Thus, the systems, methods, and computer programs of the present invention can be configured to assess a given patient over time or to assess a given patient at any point in time relative to a threshold value representing a population of patients having a particular condition. In either event, the systems, methods, and computer programs can be used to assess the efficacy of a treatment. For example, if a given sum indicates that the condition of a patient's tissue is deteriorating over time, or that it is inferior to that observed in a desirable patient pool, one can administer a genetic therapy (e.g., modified Claudin-5 AAV vector) and repeat the comparative process. If the deterioration slows or the patient's condition approaches that observed in the desirable pool, one can conclude that the therapeutic regime is effective or is having a positive effect on the patient's medical condition.

Electrical signals can be acquired from biological tissues by any means known in the art. For example, first and second electrical signals can be acquired from first and second electrocardiograms; first and second electromyograms (EMGs or simply “myograms”); or first and second electroencephalograms (EEGs). The means for acquiring the electrical signal can be selected on the basis of the medical condition being assessed. For example, while the invention is not so limited, one can use an electrocardiogram to assess heart failure and an electromyogram and/or electroencephalogram to assess neurodegenerative disease, brain damage (whether congenital or caused by trauma, substance abuse, or a disease such as Parkinson's Disease, Huntington's Disease, or Alzheimer's Disease), or other conditions. For example, the systems, methods, and computer programs described herein can be used to assess a patient who has, or who has been diagnosed as having, polymoysitis, a denervated tissue (resulting, for example, from trauma), carpal tunnel syndrome, amyotrophic lateral sclerosis (ALS), muscular dystrophy, myasthenia gravis, alcoholic neuropathy, axillary nerve dysfunction, Becker's muscular dystrophy, brachia! plexopathy, cervical spondylosis, dermatomyositis, Duchenne muscular dystrophy, Friedreich's ataxis, or Shy-Drager syndrome. An electromyogram can be used where, for example, a neurodegenerative disease causes a motor disorder. While certain methods of detecting an electrical signal provide the advantage of being non-invasive (e.g., an electrocardiogram), the invention is not so limited. Electrical signals can also be detected and transmitted by devices that are wholly or partially implanted within the patient's body.

Optionally, the systems, methods, and computer programs can exclude results of the first calculation that are greater than a first predetermined value and that are less than a second predetermined value from the average of results of the first calculation. The first predetermined value can be the sum of a mean and a standard deviation of the results of the first calculation and the second predetermined value can be the difference between the mean and the standard deviation. For example, averaging using standard deviation calculations that eliminate the top 5% and bottom 5% of the template calculations can compensate for miscellaneous anomalies. Greater or lesser percentages may be used depending upon the anomaly (e.g., one can eliminate 1%, 2%, 3%, or 4% of the top and bottom template calculations where that minimal correction neutralizes the anomaly or more than 5% of the top and bottom template calculation (e.g., 6, 8, 10, or 12%) where that extent is required for correction).

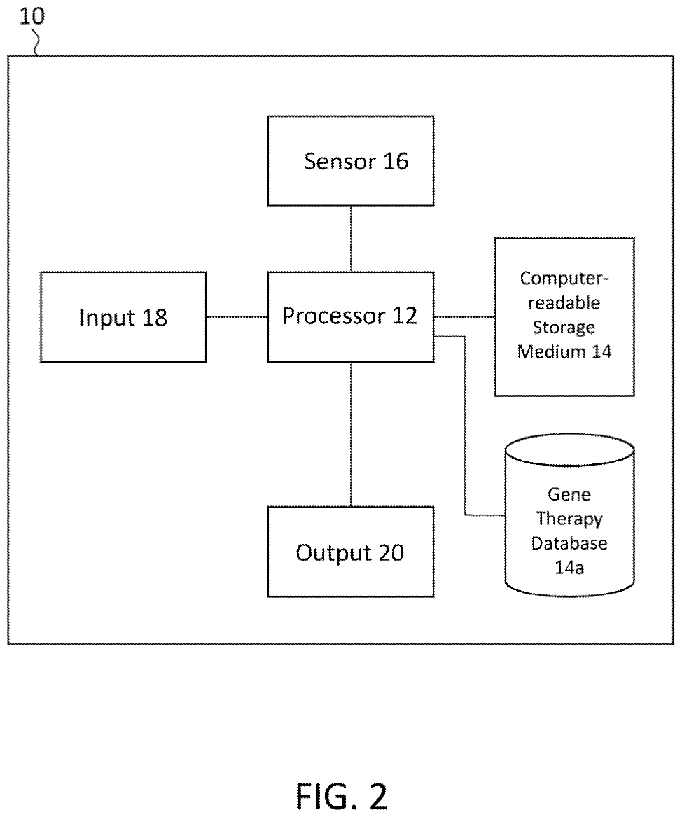

Referring to FIG. 2, in one embodiment, a system for monitoring the performance or effectiveness of an administration of a gene therapy is disclosed. The system 10 may include a processor 12 in electrical communication with a computer-readable storage medium 14. The processor 12 may also be in electrical communication with a sensor 16. The system 10 may also include an input 18 and an output 20. The input 18 may include a variety of devices known to one of skill in the art, including a mouse, keyboard, camera, microphone, scanners, etc. The output 20 may include a variety of devices known to one of skill in the art, including a display, printer, speakers, etc. Accordingly, information may be provided to the processor 12 via an input 18, a computer-readable storage medium, or a sensor 16.

The processor 12 may be configured to receive an input and perform the functions as described above. Thus, the processor 12 may be operable to determine a first baseline template corresponding to an average of results of a first calculation performed on a multiple of events within a first electrical signal acquired from the sensor 16 at a first time; (2) obtaining a first captured template corresponding to an average of results of the first calculation performed on a multiple of events within a second electrical signal acquired at a second time, which is later than the first time; (3) obtaining a second baseline template corresponding to an average of results of a second calculation performed on a multiple of events within the first electrical signal; (4) obtaining a second captured template corresponding to an average of results of the second calculation performed on a multiple of events within the second electrical signal; (5) calculating a first variance between the first baseline and captured templates; (6) calculating a second variance between the second baseline and captured templates; (7) optionally, scaling the first and second variances by first and second weighting factors, respectively, to produce first and second scaled variances; and (8) calculating a first sum of the first and second variances or, if scaled, of the scaled variances.

[000110] The system 10 is then configured to provide feedback for the user in treating a patient by administering a genetic therapy. Such feedback may include methods of administration, compounds for administration, dosages, vector identifications, relevant genes to be administered, compounds for co-delivery, and any other feedback known to one of skill in the art.

The system 10 may continue to monitor the patient during and after the administration of the gene therapy to determine the effectiveness of the gene therapy and any adjustments or follow up administrations that may be necessary. For example, the system 10, after an initial administration of the gene therapy, may receive feedback via the sensors 16 regarding protein production and determine that the transduction of the gene therapy and consequential production of proteins as measured was less than the baseline-desired production in a standard population. The system 10 then adjusts the administration of the next gene therapy according to the operating parameters and in conjunction with the data collected from previous administrations, for the patient, other patients, or both.

For example, if the system 10 detects via the sensors 16 an increased immune response during and after the administration of the gene therapy, the system may determine that a variation in the vector format may need to be altered.

A representative set of examples will be illustrated below; however, the examples are not to be construed as limiting to the scope of the system and methods described herein. Furthermore, these examples may discuss gene therapies not previously described in this application. The scope of the disclosure of a monitoring and remote monitoring system is not to be limited to the specific examples or the previous discussion of the AAV vectors and Claudin gene therapies disclosed herein. AAV has become appreciated as a good vector for the transduction of postmitotic cells. Retroviral vectors remain the vector of choice for the transduction of stem or progenitor cells despite the inherent concern of possible oncogenesis. These considerations apply for situations in which long-term transgene expression is desired. In cases such as immunization or vector-induced oncolysis, where expression at higher levels for relatively short periods of time is desirable, other viral vectors such as those derived from adenovirus and herpesvirus have more useful characteristics. Other nonviral vector systems such as lipid-mediated vectors, hydrodynamic delivery, and the gene gun are also available. What has become apparent is that different vectors have characteristics that are advantageous in specific cases. Thus, the notion of best vector depends on the question of what is best for what purpose. One of skill in the art will recognize that the system and methods disclosed herein may be applicable to any gene therapy or drug administration.

### Example 1

Diabetic nephropathy is the main cause of end-stage renal disease requiring dialysis in developed countries. The therapeutic effect of hepatocyte growth factor (“HGF”) on advanced rather than early diabetic nephropathy has been demonstrated. Early diabetic nephropathy is characterized by albuminuria, hyperfiltration, and glomerular hypertrophy, whereas advanced diabetic nephropathy shows prominent transforming growth factor (TGF)-131 upregulation, mesangial expansion, and glomerulosclerosis. An SP1017-formulated human HGF (hHGF) plasmid may be administered by intramuscular injection combined with electroporation of a patient with early and advanced diabetic nephropathy. hHGF gene therapy may upregulate endogenous HGF in the diabetic kidney. hHGF gene therapy may reduce albuminuria and induce strong regression of mesangial expansion and glomerulosclerosis in advanced diabetic nephropathy. These findings were associated with suppression of renal TGF-131 and mesangial connective tissue growth factor (“CTGF”) upregulation, inhibition of renal tissue inhibitor of metalloproteinase (TIMP)-1 expression, and reduction of renal interstitial myofibroblasts.

With this in mind, a variety of inputs may be selected for determining the efficacy and adjustments needed in the treatment of diabetic nephropathy by genetic therapies. A patient may be monitored for transforming growth factor (TGF)-131 and any changes in its regulation. These levels may be monitored via a variety of sensors including, but not limited to, microfluidic and nanofluidic chips, microarrays for electrophoresis, transcriptome analysis, PCR amplification, and any other sensor as known by one of skill in the art.

Other compounds, molecular structures, or cell structures may likewise be monitored, including HGF, myofibroblasts, mesangial connective tissue growth factor, and renal tissue inhibitor of metalloproteinase. Detection and measurements of these monitored targets may be indicative of the efficacy of the gene therapy after administration as the levels may rise or drop. Furthermore, the sensors 16 may detect an increase in lymphokines, immunoglobulins, or signal proteins indicative of an immune response after the administration of the gene therapy. The sensors 16 send a signal to the processor 12, which may determine an above-threshold immune response to the administration of the gene therapy. The processor 12 may then select a different vector from a gene therapy database 14a or the computer-readable storage medium 14. The system 10 will then provide this feedback to a user for making the determination on future administrations of a gene therapy.

### Example 2

Gene therapy in blood vessels in cases may be beneficial for patients suffering from diabetes and heart failure. The system 10 disclosed herein may be applied in a real-time remote monitoring technology in hospitals, homes, or nursing homes. Specifically, monitoring the heart consists of monitoring many types of cells, including cardiomyocytes, vascular cells, neural cells, and cardiac fibroblasts. Adult cardiomyocytes are terminally differentiated cells, and loss of cardiomyocytes as a result of heart damage is irreversible. To regenerate damaged hearts and restore cardiac function, understanding the cellular and molecular basis of heart development is of considerable importance.

Heart function is tightly regulated by cell-cell interactions. Furthermore, cell-cell interactions play an important role in heart development and function. The balance between neural chemoattractant and chemorepellents secreted from cardiomyocytes determines cardiac nervous development. Nerve growth factor is a potent chemoattractant synthesized by cardiomyocytes, whereas SEMA3A is a neural chemorepellent expressed specifically in the sub endocardium. Disruption of this molecular balance induces disorganized cardiac innervation and may lead to sudden cardiac death due to lethal arrhythmias. Cardiac fibroblasts, of which there are large populations in the heart, secrete high levels of specific extracellular matrix and growth factors. Embryonic cardiac fibroblast-specific secreted factors collaboratively promote mitotic activity of embryonic cardiomyocytes and expansion of ventricular chambers during carcinogenesis. Cardiac fibroblasts can be directly reprogrammed into cardiomyocyte-like cells in vitro and in vivo by gene transfer of cardiac-specific transcription factors.

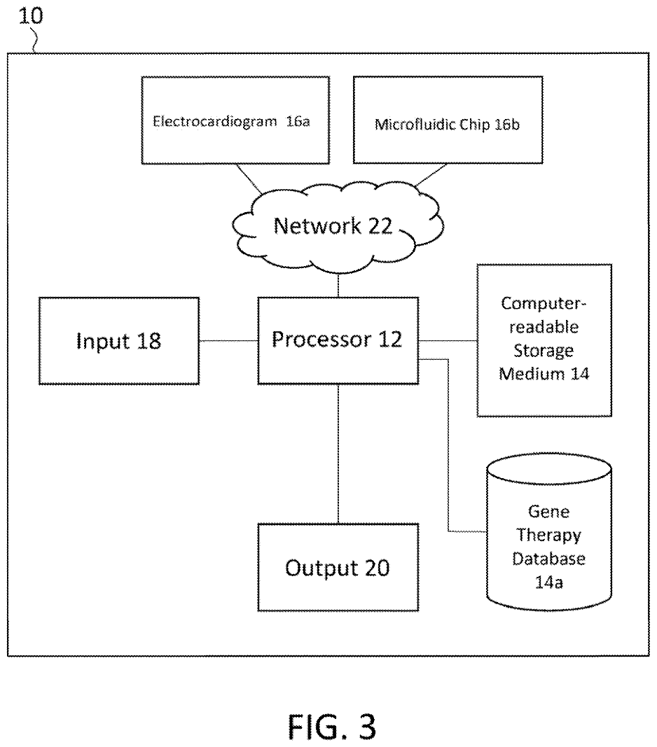

The system 10 disclosed herein may likewise be applied in monitoring the efficacy or need for gene therapy. For example, before, during, and after administration of a gene therapy, sensors 16 may be interfaced with a patient to detect certain events or materials present or expressed in or by the patient. For example, the sensors 16 may include an electrocardiogram 16a and a microfluidic chip 16b, as shown in FIG. 3. The sensors 16 may be interfaced with the patient prior to the administration of the gene therapy in order to determine a baseline reading of the patient's condition. For example, the processor 12 may receive signals from the sensors 16 reporting the electrical function of the heart as well as signal proteins, hormones, or other molecules indicative of heart failure. The system 10 may determine the baseline levels of the patient according to the method described herein and compare those known to be normal in a population.

[000122]Once the system 10 has established the baseline for the patient, the system 10 may recommend specific treatments for a patient, including specific vectors known to be effective in transducing genetic information to the damaged or diseased cardiac tissue. Likewise, the system 10 may determine specific genes for transfer to the targeted tissue. For example, the system 10 may have detected cardiac arrhythmias and decreased SEMA3A levels in the patient. The system 10 may recommend the use of a modified SEMA3A vector specifically suited for transducing sub endocardium cells.

The system may then monitor during and after the administration of the genetic therapy (the modified SEMA3A vector) to determine the efficacy of the treatment and whether adjustments or modifications may be recommend according to the operating parameters. The system 10 may be operable to communicate over a network 22 such that a patient may be monitored remotely. This allows the provider to maintain a record of the treatment and development of the disease. Thus, the sensors 16 may be operable to send the data to the processor 12 via a remote connection. In some embodiments, the sensors 16 may also be operably to encrypt the data prior to sending the data over the network 22 and the processor 12, upon receiving the data, is able to decrypt the encrypted data sent by the sensors 16. A remote system 10 is demonstrated in FIG. 3.

Although two specific examples are provided, one of skill in the art will recognize that the system and methods disclosed relating to a monitoring system may be applied in a variety of settings and for a variety of genetic therapies. A few examples are included below.

TABLE 1

| Condition | Gene |
|---|---|
| Cystic Fibrosis | CFTR |
| Canavan's Disease | Aspartoacylase |
| Parkinson's Disease | GAD65, AADC, neurturin |
| Alzheimer's Disease | Beta nerve growth factor |
| Alpha-antitrypsin deficiency | AAT |
| Arthritis | TNFR:FC |
| Leber congenital amaurosis | RPE65 |
| Hemophilia B | Factor IX |
| Late infantile neuronal | CLN2 |
| lipofuscinosis | |
| Muscular Distrophy | Minidystrophin, sarcoglycan |
| Heart Failure | SERFCA-2a |
| Prostate Cancer | Granulocyte-macrophage |
| | colony-stimulating factory |
| Epilepsy | Neruropeptide Y |

Although this is not an exhaustive list or disclosure of genetic therapies, the disclosure relates generally to systems and methods for monitoring, assessing, and improving medical conditions and genetic therapies. One of skill in the art will recognize the application of the disclosure to a variety of settings.

Thus, although there have been described particular embodiments of the present invention of new and useful GENE THERAPIES, SYSTEMS, AND METHODS FOR MONITORING, it is not intended that such references be construed as limitations upon the scope of this invention.

What is claimed is:

1. An adeno-associated virus (AAV) genome comprising:
at least one inverted terminal repeat;
a promotor; and
a Claudin-5 gene.

2. The adeno-associated virus (AAV) genome according to claim 1, further comprising:
at least two inverted terminal repeats.

3. An adeno-associated virus (AAV) genome comprising:
at least one inverted terminal repeat;
a promotor; and
a Claudin-3 gene.

4. The adeno-associated virus (AAV) genome according to claim 3, further comprising:
at least two inverted terminal repeats.

5. A method of administering a gene therapy for treating diabetic macular edema, comprising the steps of:
obtaining an adeno-associated virus (AAV) transfected with a Claudin-5 gene; and
administering the AAV subretinally.
